(19) United States
(12) Patent Application Publication
(10) Pub. No.: US 2024/0344100 A1
(43) Pub. Date: Oct. 17, 2024

(54) SELF-REPLICATING DNA EXPRESSION SYSTEM AND IMMUNOGEN

(71) Applicant: University of Cape Town, Rondebosch (ZA)

(72) Inventors: Edward Peter Rybicki, Rondebosch (ZA); Guy Louis Regnard, Rondebosch (ZA); Warren Ralph Josephus De Moor, Rondebosch (ZA)

(21) Appl. No.: 18/293,736

(22) PCT Filed: Aug. 4, 2022
(86) PCT No.: PCT/IB2022/057236

§ 371 Date: Jan. 30, 2024

(30) Foreign Application Priority Data
Aug. 4, 2021 (GB) 2111290.9

(51) Int. Cl. C12P 21/00; C12N 15/86

(52) U.S. Cl. CPC C12P 21/00; C12N 15/86; C12N 2750/10043; C12N 2750/10052

(57) ABSTRACT

The present invention relates to self-replicating DNA expression vectors comprising elements from Beak and Feather Disease Virus (BFDV), including a BFDV LIR element comprising an origin of replication (Ori), genes encoding a BFDV Rep protein and a heterologous polypeptide of interest under the control of a mammalian promoter, wherein the Rep protein is capable of binding to the Ori to initiate replication of the expression vector. Pharmaceutical compositions and cells comprising the self-replicating vectors are also provided. The self-replicating vectors may be used in methods of inducing an immune response or expressing a polypeptide in a subject, or as an expression system.

### BACKGROUND OF THE INVENTION

The present invention relates to a self-replicating DNA expression vector derived from beak and feather disease virus (BFDV) for expressing a protein of interest, comprising a BDFV LIR element which includes an origin of replication (Ori), a gene encoding the polypeptide of interest, and a rep gene encoding a viral replication-associated protein (Rep), wherein the rep gene and the gene encoding the protein of interest are under the control of a mammalian promoter, further wherein the Rep protein binds to the BFDV LIR element to initiate rolling circle replication in a mammalian host cell that amplifies the vector including the gene encoding the protein of interest to high copy number.

The recent COVID-19 pandemic has highlighted that there is a dire need for the rapid development of safe and effective vaccines that can be fast-tracked through clinical trials and easily manufactured at scale. Genetic vaccines using DNA or mRNA offer several advantages over conventional vaccines in these areas. Recently, RNA vaccines for SARS-COV-2 have demonstrated the capability of generating strong humoral and cellular immune responses. DNA vaccines, which are more stable and generate a similar kind of response, often suffer from lower levels of efficacy. One apparent reason for this is that DNA requires delivery into the nucleus to be effective, a step that incurs significant loss.

DNA vaccines provide several advantages over traditional virus- and protein-based vaccines. The ease of DNA manipulation allows for rapid development and production of candidate vaccines capable of being distributed without the need for a cold chain in cases of pandemics. In the case of influenza, it has been suggested that DNA vaccines could be used as an initial priming dose until traditionally produced, inactivated flu vaccines become available. DNA vaccines have been demonstrated to be safe and well-tolerated; however, one of the critical drawbacks has been the limited ability to produce robust immunogenicity. This is despite the benefit of endogenous recombinant expression of the antigen that facilitates natural conformation and post-translational modifications.

In multiple phase I clinical trials of DNA vaccines encoding HIV antigens, 60-70% of individuals developed detectable CD4+ T-cell responses, and less than 25% developed CD8+ T-cell responses. Similarly, a study with DNA encoding an avian influenza antigen was only capable of generating a T cell response in 20% of individuals. Poor T-cell responses are problematic, particularly with retroviral infections such as HIV-1, given the importance of the cellular response against infection and viral control. Several approaches have been or are being investigated to address low immunogenicity. These include adjuvants, alternative delivery devices, improving vector design, antigen codon optimisation, and higher and multiple doses. A multipronged approach is often suggested to boost the effectiveness of DNA vaccines.

One reason behind low immunogenicity is inefficient DNA delivery into mammalian cell nuclei in vivo. As soon as foreign DNA enters the cytoplasm, it can be recognised by a variety of pathogen recognition receptors (PRRs) which act as potent stimulators of innate and T-cell immunity. Indeed, CpG rich DNA can be used as an adjuvant to activate innate antiviral biochemical pathways and enhance the type I response. PRRs that bind foreign DNA activate the cGAS-cGAMP-STING pathway, which may be stimulated from both the cytoplasm and the nucleus to generate a broader innate antiviral pathogen-associated molecular pattern (PAMP) biochemical response. This along with DNA damage-associated molecular pattern (DAMP) signaling, generated by the active RCR episomes, will help create a unique immunogenic cellular response. As these responses directly detect pathogens through the presence of their replicating genomic material, it is reasonable to assume that increasing the concentration of foreign DNA, via use of self-replicating DNA vectors, would also amplify the strength of the innate PRR-stimulated immunological response. Indeed, it has previously been postulated that the detection of pathogen genomic replication might be a critical indicator of pathogen viability, that is used by the innate immune system to determine the level of biological threat to the cell and the host. Innate immune responses have been shown to correlate strongly with pathogen viability when inactivated and live vaccines are compared. Self-replicating DNA vaccines can mimic live pathogens in terms of their ability to stimulate the innate PRRs responsible for identifying pathogen genomic replication and gene expression.

Previous studies have looked at self-replicating geminivirus-based DNA expression systems in plants to improve heterologous protein; porcine circovirus (PCV)-based constructs have also been used in pigs to generate attenuated viruses. A chimaeric PCV-1/PCV-2 molecular clone was shown to generate infectious but attenuated virus, which produced a specific antibody response when injected in pigs. No studies in animals have explored the use of expression vector DNA replication to enhance immunogenicity. However, a DNA-launched self-replicating RNA system derived from an alphavirus has shown that low dosages could generate improved immune responses compared to a non-replicating vector.

Beak and feather disease circovirus (BFDV) is a closed circular ssDNA virus that belongs to the family Circoviridae and genus Circovirus, whose type species is Porcine circovirus 1. BFDV has a host range limited to psittacines (parrots) and is the causative agent of psittacine beak and feather disease (PBFD). BFDV is one of the smallest animal viruses (2 kb genome) and has a very simple replication strategy. Rolling circle replication (RCR) is initiated by a viral replication-associated protein (Rep) at an origin of replication (ori) situated in a non-coding region of the BFDV genome. Here a crucial DNA stem-loop structure is bound and nicked by Rep, allowing it to self-prime and recruit the activity of the host cell's DNA polymerase to carry out viral genomic replication via RCR. In this process, multiple intermediate DNA states are created that include ssDNA and dsDNA in both linear and circular forms.

The virus is non-infectious in humans; however, it can replicate its genome within mammalian cells after transfection. These features make the virus well-suited for use in developing a replicating DNA vaccine.

The inventors have developed self-replicating DNA expression vectors using replication genes and other elements from the BFDV genome, which have the potential to mitigate issues with existing DNA vaccines, including the reduced efficacy as a result of the significant loss associated with delivery into the nucleus, as well as methylation and gene silencing. These vectors employ a viral rolling circle replication cycle in mammalian host cells that amplifies vector and gene of interest (GOI) copy number in a viral-like manner while maintaining themselves as episomes. These vectors maintain persistently elevated GOI expression levels. Furthermore, they generate alterations in cellular morphology and growth rate synonymous with increased cellular stress, which acts as an immune stimulus by stimulating known DNA pathogen recognition receptors and generating innate immune responses as well as significant amounts of immunogen.

These vectors may be used for gene expression, vaccines, or as immunogens and innate immune pathway activators. The vectors may also find application in the production of difficult to express proteins/genes or peptides due to their ability to inhibit cellular apoptosis.

### SUMMARY OF THE INVENTION

The present invention relates to self-replicating DNA expression vectors derived from Beak and Feather Disease Virus (BFDV) including a gene encoding a BFDV replication-associated protein (Rep) and a gene encoding a heterologous non-BFDV polypeptide of interest, both operably linked to a mammalian promoter; and a long intergenic region (LIR) element from the BFDV comprising an origin of replication (Ori), wherein the Rep protein is capable of binding to the Ori to initiate replication of the expression vector. The invention also relates to pharmaceutical compositions and to cells comprising the self-replicating DNA expression vector described herein. The self-replicating DNA expression vector may be useful in methods of inducing an immune response in a subject and in methods of expressing a polypeptide in a subject. Thus, the self-replicating DNA expression vector of the invention may be useful as a DNA vaccine or as a DNA therapeutic. The self-replicating DNA expression vector of the invention may also be useful in an in vitro or ex vivo method of expressing a polypeptide in a cell.

According to a first aspect of the present invention there is provided for a self-replicating DNA expression vector comprising or consisting of: (a) at least one BFDV LIR element comprising an origin of replication (Ori); (b) a gene encoding a BFDV replication-associated protein (Rep); (c) a gene encoding a heterologous polypeptide of interest; and (d) a mammalian promoter operably linked to (b) and (c), wherein the Rep protein is capable of binding to the Ori to initiate replication of the expression vector.

In a first embodiment of the self-replicating DNA expression vector of the invention, the vector may comprise a vector backbone. It will be appreciated by those of skill in the art that any plasmid backbone that facilitates plasmid production may be used. By way of a non-limiting example, the vector of the invention may be produced in E. coli and comprise an E. coli plasmid backbone, such as pUK57 plasmid backbone. Further, the vector of the invention may be produced in a yeast cell and comprise a backbone useful for producing the vector in a yeast cell. In an alternative non-limiting example, the plasmid backbone may be excluded and the vector may be produced synthetically.

In a second embodiment of the self-replicating DNA expression vector of the present invention, the vector may comprise a second BFDV LIR element, optionally having the same sequence as the first BFDV LIR element.

According to a third embodiment of the self-replicating DNA expression vector of the invention, (a) and (b) may be in the same orientation on the vector, preferably (a) and (b) are in a forward orientation on the vector.

In a fourth embodiment of the self-replicating DNA expression vector of the invention, (c) and (d) may be in the same orientation on the vector, preferably (c) and (d) are in reverse orientation on the vector, and are in the opposite orientation to (a) and (b).

According to a further embodiment of the self-replicating DNA expression vector of the present invention, the mammalian promoter is a strong mammalian promoter, such as a simian vacuolating virus 40 promoter (SV40). It will be appreciated by those of skill in the art that other strong mammalian promoters known in the art may be used, for example the promoter may be a constitutive promoter including, CMV promoter (human cytomegalovirus promoter), CAG promoter (hybrid promoter containing CMV enhancer, chicken beta actin promoter, and rabbit beta-globin splice acceptor), EF1a promoter (human elongation factor 1 alpha promoter), or the promoter may be an inducible promoter such as TRE (tetracycline response element promoter).

In another embodiment of the self-replicating DNA expression vector of the invention, the vector may have a truncated or absent BFDV capsid protein gene. Preferably, the BFDV capsid protein gene is non-functional.

In an embodiment of the self-replicating DNA expression vector of the invention, the vector may further comprise one or more restriction enzyme recognition sites, to facilitate insertion of the gene encoding the heterologous polypeptide of interest.

In a further embodiment of the self-replicating DNA expression vector of the invention, the vector may further comprise a partial Kozak sequence, optionally having a nucleic acid sequence of SEQ ID NO:5. Such Kozak sequence may be included to increase gene expression of the gene encoding the heterologous polypeptide of interest. For example, the Kozak sequence may be included between promoter and the gene encoding the polypeptide of interest.

In another embodiment of the self-replicating DNA expression vector of the invention, the vector is capable of replicating to high copy number and remaining as an episome.

According to a second aspect of the present invention there is provided for a pharmaceutical composition comprising the self-replicating DNA expression vector as described herein and a pharmaceutically acceptable diluent or excipient.

In a first embodiment of the pharmaceutical composition of the invention, the pharmaceutical composition is capable of eliciting a protective immune response in a subject against a disease.

According to a second embodiment of the pharmaceutical composition of the invention, the pharmaceutical composition may be formulated for intramuscular, intradermal, intravenous or subcutaneous administration.

In a third aspect of the present invention there is provided for a cell comprising the self-replicating DNA expression vector as described herein. In a non-limiting embodiment, such cell may be used in a method of eliciting an immune response in a subject, in a method of treating and/or preventing a disease in a subject, and/or for laboratory research applications. Methods of administering the cell to a subject are further contemplated according to the present invention.

According to a fourth aspect of the present invention, there is provided for a self-replicating DNA expression vector as described herein for use in a method of inducing an immune response in a subject, the method comprising administering the self-replicating DNA expression vector to the subject. For example, the self-replicating DNA expression vector may be for use in inducing a neutralising antibody response or a cytotoxic T lymphocyte response in a subject.

In a fifth aspect of the present invention, there is provided for a self-replicating DNA expression vector as described herein for use in a method of expressing a polypeptide in a subject, the method comprising administering the self-replicating DNA expression vector to the subject, including a human subject. In a non-limiting embodiment, the polypeptide may be a therapeutic polypeptide for treating a disease or condition.

Also contemplated according an aspect of the present invention are methods of inducing an immune response, for example a neutralising antibody response or a cytotoxic T lymphocyte response, in a subject comprising administering the self-replicating DNA expression vector described herein to the subject.

According to a further aspect of the present invention, there is provided for a method of expressing a polypeptide in a subject, including a human subject, the method comprising administering the self-replicating DNA expression vector as described herein to the subject. Such polypeptides may include therapeutic polypeptides for treating a disease or condition in the subject.

In yet another aspect of the present invention, there is provided for an in vitro or ex vivo method of expressing a polypeptide in a cell comprising transfecting the cell with the self-replicating DNA expression vector described herein. In one embodiment, the method may include recovering the polypeptide from the cell and/or from a growth medium containing the cell.

### BRIEF DESCRIPTION OF THE FIGURES

Non-limiting embodiments of the invention will now be described by way of example only and with reference to the following figures:

FIG. 1: The layout of each vector used in the present study (not shown to scale). Each of the vectors is based on pUC57 RCR+/RCR− BFDV genome vectors. Restriction endonucleases used for cloning purposes are indicated. RCR+ vectors are replicating, while RCR− vectors are non-replicating. The dashed line indicates regions of no DNA sequence. SV40=simian vacuolating virus 40 promoter; luc=luciferase gene, ori=BFDV origin of replication which contains a conserved nonanucleotide motif, that is nicked by Rep to initiate RCR; rep=BFDV replication-associated protein; cp=BFDV capsid protein; CMV=cytomegalovirus promoter; ATG=disrupted start codon; Δrep=truncated rep; Δcp=truncated cp; egfp=enhanced green fluorescent protein.

FIG. 2: Vector diagram of RCR+ eGFP in a pUK57 plasmid backbone.

FIG. 3: Overexpression of co-transfected BFDV Rep rescues replication but inhibits luminescence from a vector containing BFDV replication sequences and a non-expressing rep. Panel A is a graph that shows the effect of co-transfected BFDV rep on luminescence from the pGL4.13 luciferase expression vector (positive control). Panel B is a graph that demonstrates that adding rep by co-transfecting pTHRep prevents luminescence from the RCR− LucF vector evaluated up to 90 hours post-transfection. Panel C is a gel electrophoresis image that shows that introducing rep generates RCR amplicons of 2.0 kb with the RCR− BFDV (4.5 kb), (same as RCR+ control) and 2.8 kb with RCR− LucF (5.4 kb), as predicted. A 1:10 ratio of pTHRep to RCR− LucF is used on the final lane of the gel. Transfections done in HEK293T cells. Error bars represent standard error (n=6).

FIG. 4: Reducing the co-transfection ratio of pTHRep restores luminescence from RCR− LucF. HEK293T cells were transfected, and error bars represent standard error (n=6). Filler DNA (puk19) was used to maintain total DNA transfection concentrations.

FIG. 5: Luminescence intensity in the presence and absence of the SV40 promoter upstream of the Luc reporter gene and the presence and absence of BFDV Rep supplied in trans. HEK293T mammalian cells were transfected, and error bars represent standard error (n=6). *** (P<0.0001).

FIG. 6: Expression of Rep in cis using the native viral promoter inhibits reporter gene expression in various mammalian cell lines, but this effect is mitigated in Hela S3 cells when the reporter gene is placed in the reverse orientation. Panel A provides a comparison between the replicating (RCR+ LucF) and non-replicating BFDV (RCR− LucF) vectors in HEK293T and HeLa S3 mammalian cells (n=8). Panel B provides a comparison of RCR+ LucR and RCR− LucR when the luciferase reporter gene is placed in the reverse orientation in HEK293T and HeLa S3 mammalian cells (n=3). RCR+ LucF and RCR− LucF served as additional controls. Assays were performed at 72 hours post-transfection, and error bars represent standard error. * (P<0.05).

FIG. 7: Luminescence in HeLa S3 cells transfected with replication-competent RCR+ LucR vector surpasses that of the non-replicating RCR− LucR and positive control vectors at both 96 and 120-hours post-transfection. At 96 hours, cells had been passaged once and at 120 hours, cells had been passaged twice. Error bars represent standard error (n=6). ns (not significant), * (P<0.05), ** (P<0.01).

FIG. 8: Merged fluorescence and bright-field images of eGFP fluorescence in Hela S3 cells 8 days' post-transfection. Fluorescence by cells transfected with the replication-competent vector (RCR+ eGFPR) have greater fluorescence intensity and rounder cell morphology than cells transfected with the non-replicating vector (RCR− eGFPR). Arrows (white) indicate fluorescing cells in RCR− eGPFR control.

FIG. 9: Analysis of confocal microscopy data demonstrates that HeLa S3 cells transfected with the replication-competent BFDV expression vector (RCR+eGFPR, N=86) are larger in size (P<0.0001) and have greater mean eGFP fluorescence and cell size (P<0.0001) compared to non-replicating vector-transfected cells (RCR− eGFPR, N=115) at 24 hours post transfection. Error bars represent 95% confidence interval of the overall mean.

FIG. 10: Cross section examples of the anterior tibialis muscle of Balb/cJ mice in green channel for eGFP. Inoculated with: A) pTHRep only (Neg control), B) RCR− eGFP, C) RCR+ eGFP and D) RCR+ eGFP and pTHRep.

FIG. 11: Merged tile scan images cross sections of anterior tibialis muscle of Balb/cJ mice showing the green capture data. Inoculated with: A) pTHRep only (Neg control), B) RCR− eGFP, C) RCR+ eGFP and D) RCR+ eGFP and pTHRep.

FIG. 12: Relative change in fluorescence intensity in muscle fibre bundles of Balb/cJ mice. The mice were inoculated with 50 μg of either RCR+ or RCR− eGFP expression plasmid and 10 μg pTHRep plasmid, as indicated, in 50 μl sterile PBS. Negative control was inoculated with PBS only. (Error bars=standard error of 3 sample mean & Neg control=2σ).

### SEQUENCE LISTING

The nucleic acid sequences listed in the accompanying sequence listing are shown using standard letter abbreviations for nucleotide bases, and the standard three letter abbreviations for amino acids. It will be understood by those of skill in the art that only one strand of each nucleic acid sequence is shown, but that the complementary strand is included by any reference to the displayed strand. In the accompanying sequence listing:
SEQ ID NO:1—nucleotide sequence of BFDV LIR element (Ori).
SEQ ID NO:2—nucleotide sequence of rep gene.
SEQ ID NO:3—amino acid sequence of Rep protein.
SEQ ID NO:4—nucleotide sequence of SV40 promoter.
SEQ ID NO:5—nucleotide sequence of partial Kozak sequence.
SEQ ID NO:6—nucleotide sequence of BFDV rep sense primer.
SEQ ID NO:7—nucleotide sequence of BFDV rep antisense primer.
SEQ ID NO:8—nucleotide sequence of egfp sense primer.
SEQ ID NO:9—nucleotide sequence of egfp antisense primer
SEQ ID NO:10—nucleotide sequence of BFDV LIR element (Ori) binding motif.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items. It is, however, contemplated as a specific embodiment of the present disclosure that the term “comprising” encompasses the possibility of no further members being present, i.e., in some embodiments “comprising” is to be understood as having the meaning of “consisting of”.

The present invention, in its broadest sense, relates to chimeric Beak and Feather disease virus (BFDV) derived DNA expression vectors capable of undergoing self-replication. The inventors have shown that the self-replicating vectors described herein are capable of self-regulating in terms of both gene expression and DNA amplification levels.

The inventors have constructed self-replicating DNA expression vectors, which have the potential to mitigate many of the issues faced by DNA vaccines and gene expression systems, such as loss from biological degradation and/or inactivation from methylation or gene silencing mechanisms and innate cellular responses. These vectors employ a viral rolling circle replication (RCR) cycle within host mammalian cells that amplifies the DNA vector or a select fragment of DNA containing key genetic elements used by the system and a gene of interest (GOI). This has resulted in the development of a dynamic gene expression system that increases both gene of interest expression levels and gene copy number. Replicating DNA vaccines offer several advantages over conventional DNA vaccines. These include amplifying recombinant gene expression by increasing gene copy number, extending the half-life of DNA activity and presence within cells and sustaining antigen expression over time, thereby increasing total overall antigen exposure to the immune system. The vectors of the present invention result in rapid DNA amplification and increased antigen expression, in a manner that resembles acute viral infection at the subcellular level. Thus, the vectors of the present invention have potential to enhance the activation of innate antiviral immune responses due to these unique characteristics. Furthermore, the accumulation of amplicon DNA, which transitions through various DNA states during RCR, should stimulate DNA damage recognition receptors to generate a strong DNA damage-associated molecular pattern (DAMP) response. DAMP signalling combined with pathogen recognition receptor (PRR) stimulation has been reported to strengthen pathogen-associated molecular patterns (PAMPs) and enhance activation of innate antiviral immune responses.

In terms of safety, the absence of the BFDV capsid protein in these replicating DNA vectors effectively confines ‘replication’ specifically to DNA only and this may only occur within the cells that initially took up the DNA vector. As the RCR episomes lack the ability to relocate outside of the nucleus or to the extracellular environment and lack any protection from DNA degradation which occurs outside the nucleus, the RCR episomes are considered extremely safe and are expected to be destroyed entirely upon host cell death, which is all but guaranteed, by the destructive pathogenic nature the viral RCR cycle within a host cell that was observed in cell culture, as found by the inventors. The increasingly destructive nature of the RCR episomes, which promotes cell death, substantially differentiates RCR expression systems from non-replicating DNA vectors where the possibility of oncogenic genome integration, albeit unlikely, has been a concern.

The self-replicating vectors of the present invention have been optimised to function in a more viral like manner for immunological inducing applications such as cancer vaccines and pathogenic vaccines, or the production of difficult to express or toxic proteins in cell culture. In particular, the inventors have surprisingly found that some BFDV-derived replicating vectors include genes or express products that acted as a negative feedback mechanism on protein expression. The inventors of the present invention have constructed vectors that overcome these negative feedback mechanisms in order to replicate to high copy number and ensure high levels of expression of a polypeptide of interest.

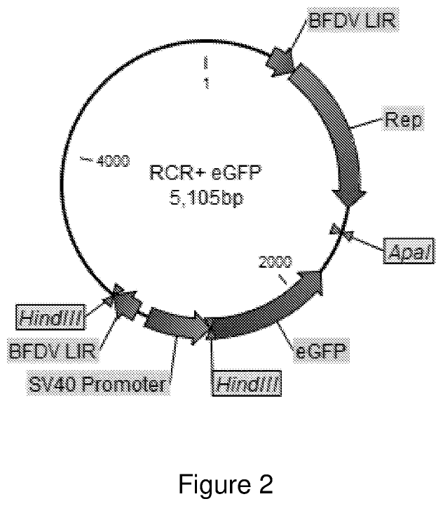

In one embodiment the self-replicating vector of the present invention includes a BFDV large intergenic region (LIR) element comprising an origin of replication (ori); followed immediately by the BFDV rep gene in a forward orientation; a gene of interest to be expressed; a powerful mammalian promoter element in reverse orientation to Rep, with a restriction site that incorporates a partial Kozak sequence to maximise gene of interest expression levels and facilitate easy addition of a gene of interest; a second LIR element in the same orientation as the first LIR element and the rep gene to allow for the removal of the plasmid backbone for maximal amplification production and stability and minimal size (FIG. 2).

The self-replicating DNA vector of the present invention may be used in several different ways. In one embodiment, the DNA vector may be used as an immunogenic vaccine, for example against pathogenic diseases and cancers. The self-replicating nature of self-replicating DNA vector enables the amplification of gene expression and mitigates against genetic silencing and normal biological losses that have hindered the successful use of non-replicating DNA expression systems as gene therapies or vaccines. As a cancer vaccine the self-replicating nature may also activate innate cellular immunological and antiviral defence mechanisms that are key in eliciting immunological responses to cancerous cell lines. Only self-replicating DNA can achieve this type of cellular effect, which non-replicating DNA vaccines cannot. Self-replication enables localised increases in expression levels of a gene of interest at the individual cellular level which cannot be achieved by non-replicating DNA expression systems. Enabling excessive antigen or gene of interest expression at the cellular level is critical in overwhelming the host cell and triggering a powerful innate cellular immune response. Using the viral-like nature of the self-replicating DNA vector of the present invention may generate strong T-cell and antibody responses. Thus, the self-replicating DNA vector may be suitable as a rapidly deployable, cheap to manufacture and scalable vaccine in response to future pandemics or as a targeted cancer vaccine for inducing innate immune responses in cancerous cells that have otherwise evaded eliciting an immune response.

In a further embodiment of the invention, the self-replicating DNA vector is suitable as an enhanced gene expression system for the manufacture of proteins or peptides of interest in mammalian cell culture. The inventors have demonstrated that the self-replicating DNA can hinder cellular apoptosis that plagues protein production attempts, particularly when the target protein is slightly toxic to the cell or when the target protein becomes toxic at high concentration levels. The self-replicating DNA vector's ability to both amplify cellular expression levels, while simultaneously suppressing cellular apoptosis, make it suitable for complex and advanced protein expression and manufacturing applications by potentially significantly improving production yields.

A “protein,” “peptide” or “polypeptide” is any chain of two or more amino acids, including naturally occurring or non-naturally occurring amino acids or amino acid analogues, irrespective of post-translational modification (e.g., glycosylation or phosphorylation).

The terms “nucleic acid” or “nucleic acid molecule” and “polynucleotide” are used herein interchangeably and encompass both ribonucelotides (RNA) and deoxyribonucleotides (DNA), including cDNA, genomic DNA, and synthetic DNA. The nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides.

The term “heterologous polypeptide of interest” or “polypeptide of interest” as used herein refers to any polypeptide that does not occur naturally in a Beak and Feather Disease Virus (BFDV). A heterologous polypeptide of interest may thus include protozoal, bacterial, viral, fungal or animal proteins. The heterologous polypeptide of interest is intended for expression in a mammalian cell according to a non-limiting embodiment of the present invention. Preferably, the mammalian cell is a non-native mammalian cell i.e., mammalian host cells other than cells from psittacines (parrots). Non-limiting examples of heterologous polypeptides of interest may include, pharmacological polypeptides (e.g., for medical uses, for cell- and tissue culture) or industrial polypeptides (e.g. enzymes, growth factors) that can be produced using the self-replicating DNA vectors of the present invention. The self-replicating DNA vectors including a gene encoding a heterologous polypeptide of interest, such as an antigen, may be useful as vaccines. Alternatively, the self-replicating DNA vectors may be used to produce heterologous polypeptides of interest in an expression system, which may be recovered and used in a vaccine, or in other reagents or diagnostics.

As used herein the terms “host mammalian cell”, “mammalian host cell” and “mammalian cell” refer to a cell of a mammal. In one embodiment the mammalian cell may be a human cell. In an alternative embodiment the mammalian cell may be an animal cell. Preferably, the animal cell is not from the native host for BFDV, in particular psittacines (parrots).

The terms “heterologous gene of interest” or “gene of interest” as used herein refers to a nucleic acid that encodes a heterologous polypeptide of interest as defined herein. Such gene of interest may be codon-optimised for expression in a mammalian cell.

The term “complementary” refers to two nucleic acids molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” sequence is an amino acid or nucleotide sequence that differs from a reference sequence only by one or more conservative substitutions, or by one or more non-conservative substitutions, deletions, or insertions located at positions of the sequence that do not destroy or substantially reduce the antigenicity of one or more of the expressed polypeptides or of the polypeptides encoded by the nucleic acid molecules. Alignment for purposes of determining percent sequence identity can be achieved in various ways that are within the knowledge of those with skill in the art. These include using, for instance, computer software such as ALIGN, Megalign (DNASTAR), CLUSTALW or BLAST software.

Those skilled in the art can readily determine appropriate parameters for measuring alignment, including any algorithms needed to achieve maximal alignment over the full length of the sequences being compared. In one embodiment of the invention there is provided for a polypeptide or polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Those skilled in the art will appreciate that polypeptides, peptides or peptide analogues can be synthesised using standard chemical techniques, for instance, by automated synthesis using solution or solid phase synthesis methodology. Automated peptide synthesisers are commercially available and use techniques known in the art. Polypeptides, peptides and peptide analogues can also be prepared from their corresponding nucleic acid molecules using recombinant DNA technology.

In some embodiments, the nucleic acid molecules of the invention may be operably linked to other sequences. By “operably linked” is meant that the nucleic acid molecules encoding the Rep protein and/or heterologous polypeptides of interest are connected to regulatory sequences in such a way as to permit expression of the proteins when the appropriate molecules are bound to the regulatory sequences. Such operably linked sequences may be contained in vectors or expression constructs which can be transfected into host cells or delivered to a subject for expression.

The term “recombinant” means that something has been recombined. When used with reference to a nucleic acid construct the term refers to a molecule that comprises nucleic acid sequences that are joined together or produced by means of molecular biological techniques. The term “recombinant” when used in reference to a protein or a polypeptide refers to a protein or polypeptide molecule which is expressed from a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Accordingly, a recombinant nucleic acid construct indicates that the nucleic acid molecule has been manipulated using genetic engineering, i.e. by human intervention. Recombinant nucleic acid constructs may be introduced into a host cell by transformation or delivered to a subject using delivery mechanisms known in the art. Such recombinant nucleic acid constructs may include sequences derived from the same host cell species or from different host cell species.

The term “vector” refers to a means by which polynucleotides or gene sequences can be introduced into a cell. In particular, the vector of the present invention is derived from BFDV and is a self-replicating DNA vector.

In some embodiments, the self-replicating DNA vector of the invention may include, without limitation, at least one BFDV LIR element (SEQ ID NO:1) which comprises an origin of replication (Ori) to which Rep protein is capable of binding to initiate replication, a rep gene (SEQ ID NO:2) encoding Rep protein (SEQ ID NO:3), a mammalian promoter, such as a SV40 promoter (SEQ ID NO:4), or sequences substantially identical thereto. The sequence of the gene encoding the Rep protein may be codon-optimised for expression in a mammalian cell. The vector may further include one or more restriction sites to facilitate the addition of a gene encoding a polypeptide of interest. In a further embodiment, at least one restriction site includes a partial Kozak sequence of SEQ ID NO:5 to maximise expression levels as described herein.

Generally, polynucleotides or gene sequences are introduced into a vector by means of a cassette. The term “cassette” refers to a polynucleotide or gene sequence that is expressed from a vector, for example, the polynucleotide or gene sequences encoding the Rep protein and/or heterologous polypeptides of interest. A cassette generally comprises a gene sequence inserted into a vector, which in some embodiments, provides regulatory sequences for expressing the polynucleotide or gene sequences. In other embodiments, the vector provides the regulatory sequences for the expression of the Rep protein and/or heterologous polypeptides of interest. In further embodiments, the vector provides some regulatory sequences and the nucleotide or gene sequence provides other regulatory sequences. “Regulatory sequences” include but are not limited to promoters, transcription termination sequences, enhancers, splice acceptors, donor sequences, introns, ribosome binding sequences, poly(A) addition sequences, and/or origins of replication.

The term “promoter” refers to a DNA sequence that is capable of controlling the expression of a nucleic acid coding sequence or functional RNA. A promoter may be based entirely on a native gene or it may be comprised of different elements from different promoters found in nature. Different promoters are capable of directing the expression of a gene in different cell types, or at different stages of development, or in response to different environmental or physiological conditions. A “constitutive promoter” is a promoter that direct the expression of a gene of interest in most host cell types most of the time.

As used herein a “pharmaceutically acceptable carrier” or “excipient” includes any and all antibacterial and antifungal agents, coatings, dispersion media, solvents, isotonic and absorption delaying agents, and the like that are physiologically compatible. A “pharmaceutically acceptable carrier” may include a solid or liquid filler, diluent or encapsulating substance which may be safely used for the administration of the self-replicating DNA vectors and/or vaccine compositions of the invention to a subject. The pharmaceutically acceptable carrier can be suitable for intramuscular, intraperitoneal, intravenous, subcutaneous, oral or sublingual administration. Pharmaceutically acceptable carriers further include sterile aqueous solutions, dispersions and sterile powders for the preparation of sterile solutions. The use of media and agents for the preparation of pharmaceutically active substances is well known in the art. Where any conventional media or agent is incompatible with the active compound, use thereof in the pharmaceutical compositions of the invention is not contemplated. Supplementary active compounds can also be incorporated into the compositions.

Suitable formulations or compositions to administer the self-replicating DNA vectors and/or vaccine compositions to subjects who are to be prophylactically treated for an infection or disease, who are suffering from an infection or disease or subjects which are presymptomatic for a condition associated with infection and/or disease fall within the scope of the invention. Any appropriate route of administration may be employed, such as, parenteral, intravenous, subcutaneous, intramuscular, intracranial, intraorbital, ophthalmic, intraventricular, intracapsular, intraspinal, intrathecal, intracistemal, intraperitoneal, intranasal, aerosol, topical, or oral administration.

As used herein the term “subject” includes mammals. In one embodiment the subject may be a human. In an alternative embodiment the subject may be an animal, including livestock. In some embodiments, the term “subject” may exclude reference to the native host for BFDV, in particular psittacines (parrots).

For vaccine formulations and pharmaceutical compositions, an effective amount of the self-replicating DNA vectors and/or vaccine compositions of the invention can be provided, either alone or in combination with other compounds, with immunological adjuvants, for example, aluminium hydroxide dimethyldioctadecylammonium hydroxide or Freund's incomplete adjuvant. The self-replicating DNA vectors and/or vaccine compositions of the invention may also be linked with suitable carriers and/or other molecules, such as bovine serum albumin or keyhole limpet hemocyanin in order to enhance immunogenicity. The self-replicating DNA vectors and/or vaccine compositions of the present invention can be provided either alone or in combination with other compounds in a composition (for example, nucleic acid molecules, small molecules, peptides, or peptide analogues), in the presence of a liposome, an adjuvant, or any carrier, such as a pharmaceutically acceptable carrier and in a form suitable for administration to mammals, for example, humans, or other animals, including cattle, sheep, etc.

In some embodiments, the self-replicating DNA vectors and/or vaccine compositions produced according to the method of the invention may be provided in a kit, optionally with a carrier and/or an adjuvant, together with instructions for use.

An “effective amount” of a self-replicating DNA vectors and/or vaccine compositions according to the invention includes a therapeutically effective amount, immunologically effective amount, or a prophylactically effective amount. A “therapeutically effective amount” refers to an amount effective, at dosages and for periods of time necessary, to achieve the desired therapeutic result, such as treatment of a disease or infection, including a viral infection, or a condition associated with such disease or infection. The outcome of the treatment may for example be measured by a decrease in viremia, inhibition of viral gene expression, delay in development of a pathology associated with the infection or disease, stimulation of the immune system, or any other method of determining a therapeutic benefit. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects.

The dosage of any of the self-replicating DNA vectors and/or vaccine compositions of the present invention will vary depending on the symptoms, age and body weight of the subject, the nature and severity of the disorder to be treated or prevented, the route of administration, the disease and/or infection being treated and the form of the composition. Any of the compositions of the invention may be administered in a single dose or in multiple doses. The dosages of the self-replicating DNA vectors and/or vaccine compositions of the invention may be readily determined by techniques known to those of skill in the art or as taught herein.

By “immunogenically effective amount” is meant an amount effective, at dosages and for periods of time necessary, to achieve a desired immune response. The desired immune response may include stimulation or elicitation of an immune response, for instance a T or B cell response.

A “prophylactically effective amount” refers to an amount effective, at dosages and for periods of time necessary, to achieve a desired prophylactic result, such as prevention of onset of a condition associated with a disease and/or infection. Typically, a prophylactic dose is used in subjects prior to or at an earlier stage of disease and/or infection, so that a prophylactically effective amount may be less than a therapeutically effective amount.

Dosage values may vary with the severity of the condition to be alleviated. For any particular subject, specific dosage regimens may be adjusted over time according to the individual need and the judgment of the person administering or supervising the administration of the self-replicating DNA vectors and/or vaccine compositions of the invention. Dosage ranges set forth herein are exemplary only and do not limit the dosage ranges that may be selected. It may be advantageous to formulate the compositions in dosage unit forms for ease of administration and uniformity of dosage.

The term “preventing”, when used in relation to an infectious disease, or other medical disease or condition, is well understood in the art, and includes administration of a composition which reduces the frequency of or delays the onset of symptoms of a condition in a subject relative to a subject which does not receive the composition. Prevention of a disease includes, for example, reducing the number of diagnoses of the infection in a treated population versus an untreated control population, and/or delaying the onset of symptoms of the infection in a treated population versus an untreated control population.

The term “prophylactic or therapeutic” treatment is well known to those of skill in the art and includes administration to a subject of one or more of the compositions of the invention. If the composition is administered prior to clinical manifestation of the unwanted condition (e.g., disease or other unwanted state of the subject) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

Toxicity and therapeutic efficacy of compositions of the invention may be determined by standard pharmaceutical procedures in cell culture or using experimental animals, such as by determining the LD₅₀ and the ED₅₀. Data obtained from the cell cultures and/or animal studies may be used to formulating a dosage range for use in a subject. The dosage of any composition of the invention lies preferably within a range of circulating concentrations that include the ED₅₀ but which has little or no toxicity. The dosage may vary within this range depending upon the dosage form employed and the route of administration utilized. For self-replicating DNA vectors and/or vaccine compositions of the present invention, the therapeutically effective dose may be estimated initially from cell culture assays.

The invention also relates in part to a method of eliciting an immune response in a subject comprising administering to a subject in need thereof a prophylactically effective amount of the self-replicating DNA vectors and/or vaccine compositions of the present invention.

The sequences used in the construction of the self-replicating DNA vectors of the present invention are provided in Table 1 below. The sequence of the Rep protein is also provided in Table 1.

TABLE 1
Sequences used in construction of the self-replicating DNA vector.

| SEQ ID NO and | |
|---|---|
| Description | Sequence |
| SEQ ID NO: 1- | AGAGGTGCCCCACAGGCGGCGGTTAGTATTACCCGCCGCCTGGG |
| nucleotide | GCACCGGGGCACCGCAGCCATTGGCTGCCGTGCCGAGGTGCCC |
| sequence of BFDV | CGCCTTAGGGAGGAGTAAATGGCGCCGTTAAACGGTGCCGTAAT |
| LIR element (Ori) | TTCCGGAGGATCACAGTCGCCCGGGAACA |
| SEQ ID NO: 2- | ATGCCGTCCAAGGAGGGCTCTGGCTGTCGCCGTTGGTGTTTCAC |
| nucleotide | CCTTAACAACCCTACAGACGGCGAGATCGAATTCGTCCGTACTCT |
| sequence | CGGGCCTGACGAATTCTACTATGCCATCGTTGGACGGGAAAAGG |
| of rep gene | GCGAGCAAGGTACCCCCCATTTGCAAGGCTACTTTCATTTCAAAA |
| | ATAAGAAGCGACTGAGCGCGCTTAATAAAATGCTGCCGCGAGCTC |
| | ATTTTGAGCGCGCTAAAGGGAGTGATGCAGATAATGAGAAGTATT |
| | GCAGTAAAGAGGGGGACGTTATACTTACCCTGGGCATTGTGGCG |
| | AGAGATGGTCACCGCGCTTTCGACGGAGCTGTTGCTGCCGTGAT |
| | GTCCGGACGCAAAATGAAGGAAGTCGCGCGAGAGTTCCCAGATA |
| | TCTACGTCAGGCATGGGGGGGGCTTGCATAACCTCTCTCTATTGG |
| | TCGGTTCCCGCCCACGTGATTTCAAGACAGAAGTTGACGTCATCT |
| | ACGGGCCTCCTGGGTGTGGCAAGAGTAAATGGGCCAATGAGCAG |
| | CCTGGGACTAAATATTATAAAATGCGCGGTGAATGGTGGGATGGA |
| | TATGATGGGGAAGATGTTGTCATATTGGACGACTTTTATGGGTGG |
| | CTACCTTATTGTGAGATGCTCCGCCTTTGCGACCGTTATCCACATA |
| | AAGTGCCAGTTAAGGGCGCTTTTGTGGAGTTTACCAGCAAGAGGA |
| | TCATTATCACGAGCAATAAGTCCCCCGAGACCTGGTACAAGGAGG |
| | ACTGTGACCCGAAGCCACTGTTCCGGAGATTCACTCGTGTTTGGT |
| | GGTACACTGACAAGTTGGAACAAGTCCGGCCTGATTTCCTTGCCC |
| | ACCCCATCAATTTTTGA |
| SEQ ID NO: 3- | MPSKEGSGCRRWCFTLNNPTDGEIEFVRTLGPDEFYYAIVGREKGE |
| amino acid | QGTPHLQGYFHFKNKKRLSALNKMLPRAHFERAKGSDADNEKYCSK |
| sequence of Rep | EGDVILTLGIVARDGHRAFDGAVAAVMSGRKMKEVAREFPDIYVRHG |
| protein | RGLHNLSLLVGSRPRDFKTEVDVIYGPPGCGKSKWANEQPGTKYYK |
| | MRGEWWDGYDGEDVVILDDFYGWLPYCEMLRLCDRYPHKVPVKGA |
| | FVEFTSKRIIITSNKSPETWYKEDCDPKPLFRRFTRVWWYTDKLEQV |
| | RPDFLAHPINF* |
| SEQ ID NO: 4- | CTGAGGCGGAAAGAACCAGCTGTGGAATGTGTGTCAGTTAGGGT |
| nucleotide | GTGGAAAGTCCCCAGGCTCCCCAGCAGGCAGAAGTATGCAAAGC |
| sequence of SV40 | ATGCATCTCAATTAGTCAGCAACCAGGTGTGGAAAGTCCCCAGGC |
| promoter | TCCCCAGCAGGCAGAAGTATGCAAAGCATGCATCTCAATTAGTCA |
| | GCAACCATAGTCCCGCCCCTAACTCCGCCCATCCCGCCCCTAACT |
| | CCGCCCAGTTCCGCCCATTCTCCGCCCCATGGCTGACTAATTTTT |
| | TTTATTTATGCAGAGGCCGAGGCCGCCTCGGCCTCTGAGCTATTC |
| | CAGAAGTAGTGAGGAGGCTTTTTTGGAGGCCTAGGCTTTTGCAAA |

The following examples are offered by way of illustration and not by way of limitation.

### Example 1

### Cloning and Transfection

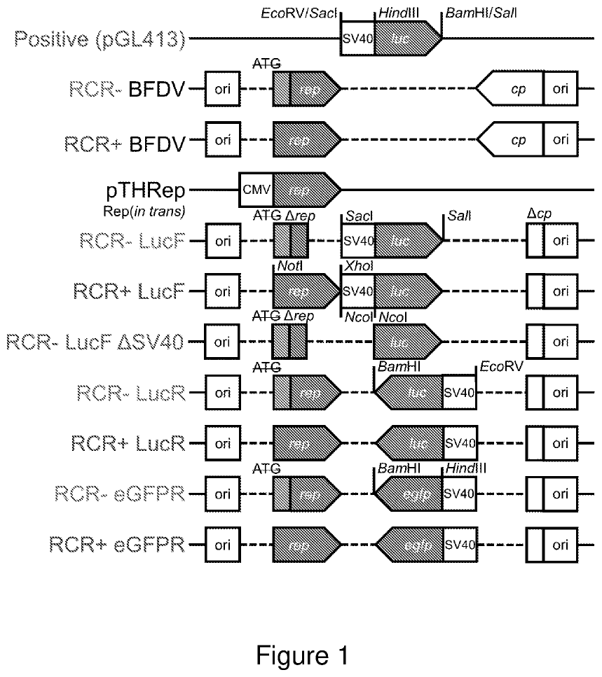

The vectors used in this study (FIG. 1) were based on a partial genome repeat of BFDV that was either capable of rolling circle replication (RCR+) or had been mutated to be non-replicative (RCR−) as described in Regnard et al (2017), which is incorporated herein by reference in its entirety. The use of an expression vector with the CMV promoter to drive Rep expression in trans was also investigated. The pGL4.13 vector (Promega Corporation) with an expression cassette containing a luc reporter gene and a SV40 early enhancer/promoter served as a positive control in all luciferase assay experiments. The SV40 enhancer has previously been shown to facilitate DNA transportation into the nucleus of cells via mechanisms described (Bai et al, 2017).

Replicating (RCR+) and non-replicating (RCR−) vectors with luciferase and enhanced green fluorescent protein (eGFP) as reporter genes were constructed using standard cloning techniques. Primers used for cloning are provided in Table 2 below. The sequence of the gene encoding the eGFP was codon-optimized for expression in mammalian cells. A vector diagram of RCR+ eGFP is provided in FIG. 2.

Luciferase expression was evaluated with luc on both the sense; forward (F) and antisense; reverse (R) DNA strands with respect to rep. All vector constructs were made in overnight E. coli DH5a cultures, and high-quality plasmid DNA was isolated using a ZymoPURE™ II DNA maxiprep kit.

DNA used for transfections was >95% supercoiled, endotoxin-free and supplied by Aldevron (USA) or prepared using a ZymoPURE™ II Plasmid Maxiprep Kit (Zymogen) as per manufacturer's instructions and evaluated by agarose gel electrophoresis. Cell lines were obtained from the American Type Culture Collection (ATCC, USA); these included HEK-293 (ATCC® CRL-1573™), HEK-293T (ATCC® CRL-3216™) and HeLa S3 (ATCC® CCL-2.2™). All cell lines were maintained at 37° C. in a 5% CO2 with 80-95% humidity incubator. The nutrient medium was exchanged every 2-3 days with DMEM (high glucose, GlutaMAX™) supplemented with 10% Gibco™ Fetal Bovine Serum (Thermo Fisher Scientific, USA) and 1% penicillin/streptomycin (100×; Sigma-Aldrich).

Cells were transfected with DNA plasmids by seeding 6, 12, or 24 well plates at a 30-50% confluency and cultured to ˜ 60-80% confluency. The transfection reagent used was X-tremeGENE™ HP DNA (Roche) and was optimised per cell line as per manufacturer's instructions. Luciferase assays were repeated multiple times using six replicates. Where comparisons of luminescence from different vectors were performed the vectors were transfected in equal copy number, calculated as pMoles and overall transfection concentration maintained by adding empty pUk19 vector as filler DNA. Where limited DNA transfection was desired, DNA transfection was terminated after 4 h by aspirating the growth media containing the transfection mix, washing 3× with PBS, and adding fresh growth media.

TABLE 2
Primers used for cloning BFDV rep and egfp.

| | | Sense Primer | | Antisense Primer | |
|---|---|---|---|---|---|
| Product | Size | Sequence | Cloning | Sequence | Cloning |
| Description | (bp) | (5′-3′) | site | (5′-3′) | site |
| BFDV rep | 1053 | GGTGGTGCGG | NotI | GGTGGTCTCG | XhoI |
| | | CCGCAGAGGT | | AGTCAAAAATT | |
| | | GCCCCACAGGC | | GATGGGGTG (SEQ | |
| | | (SEQ ID NO: 6) | | ID NO: 7) | |
| egfp | 744 | GTGGTAAGCTT | HindIII | GGTGGTGGAT | BamHI |
| | | ATGGTGAGCAA | | CCTTACTTGTA | |
| | | GGGCGAG | | CAGCTCGTCCATGC | |
| | | (SEQ ID NO: 8) | | (SEQ ID NO: 9) | |

### Analysis of Reporter Gene Expression

Total DNA was extracted using a DNeasy Blood & Tissue kit (Qiagen) as per manufacturer's instructions for cultured cells. To enrich for circular DNA, a rolling circle amplification (RCA) method was used as described by Shepherd et al (2005) with a TempliPhi amplification kit (GE Healthcare).

The luciferase assays were performed using the Luciferase Assay System (Promega) as per manufacturer's instructions. Luminescence was measured at 48 hours (h) post-transfection (pt) unless otherwise stated.

Transfections in tissue culture using fluorescent vectors were regularly inspected and imaged using a conventional Axio Vert.A1 inverted microscope (ZEISS, Germany) with 5×, 10×, 20× and 56× objective lenses, a HBO 50 halogen lighting system and a Axiocam ICm1 camera. The exposure was adjusted such that the brightest fluorescent cells were within the camera's sensitivity range.

Confocal microscopy was performed using pre-treated (0.03% poly-L-lysine in PBS for 5 minutes) four-well chamber slides. Transfections were performed using standard protocols. At 24 h or 48 h post-transfection (pt) the media was carefully aspirated with a 1 mL pipette. Cells were fixed using 100 μL of a 4% paraformaldehyde solution for 5 minutes. After fixation, cells were washed three times with PBS and stained for 30 seconds with Hoechst 33342 (Sigma, 10 mg/mL) in PBS at a ratio of 1:5000. The slides were again washed three times and thereafter treated with 20 μL Mowiol (Sigma) combined with 20 mg/mL n-propyl gallate, a cover slide was applied, and slides were dried overnight at room temperature then stored at 4° C. A LSM 880 with Airyscan inverted confocal microscope (ZEISS), equipped with an argon laser, providing light at 488 nm for eGFP fluorescence and 405 nm for Hoechst stains was used. The system used a high sensitivity photomultiplier tube imager that was calibrated against the brightest positive fluorescent sample to prevent sensor overexposure. Images were captured as a series of Z-stacks and merged into a maximum intensity projection. This allowed for the quantification of fluorescence intensity using the Zen Blue 2.5 software suite (ZEISS). Cells positive for fluorescence were identified, and the mean level of eGFP fluorescence and total surface area determined.

### Example 2

The Effect of BFDV Rep Expressed in Trans and Provided in Cis with its Native Promoter on Recombinant Protein Expression

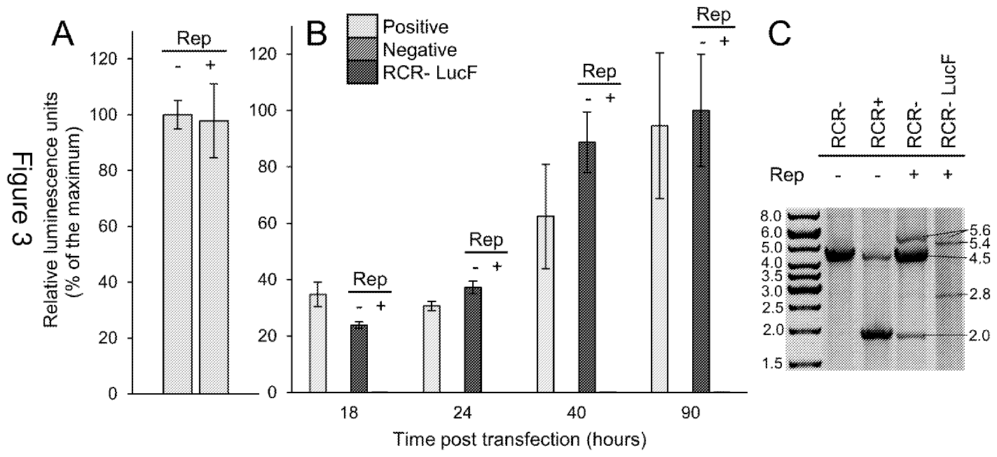

To investigate the effect of Rep expressed in trans on recombinant protein expression, a luciferase reporter gene assay was performed comparing pGL4.13 in the presence or absence of co-transfected Rep (FIG. 3A). pGL4.13 served as a positive control in all subsequent experiments. No difference in luminescence was observed in the presence or absence of Rep with pGL4.13.

Luminescence was used to investigate the effect of the BFDV ori on recombinant protein expression. The pGL4.13 luc expression cassette was placed on the sense strand (F) between two ori sequences and, in the process, created a truncated rep that had previously had the ATG disrupted (RCR− LucF vector; FIG. 3 B). The ori had no overall significant effect on luminescence compared to the positive control at 90 hours; however, it may have slightly enhanced expression at 24 h and 40 h post-transfection. When Rep was added in trans, luminescence appears almost to be wholly inhibited from 18 h post-transfection.

RCA assays demonstrated that in the presence of Rep the RCR− LucF vector (5.4 kb) formed an RCR− produced amplicon at the predicted size of 2.8 kb. This result was similar to RCR− BFDV control which also generated an RCR amplicon when Rep was added in trans (FIG. 3 C). These results indicate that, while RCR was demonstrably occurring and producing amplicons of the predicted size (FIG. 3 C), these amplicons did not appear to be increasing luciferase expression, as expected. Indeed, the precise opposite was observed; luminescence was significantly decreasing when replication was induced (FIG. 3 B). Furthermore, any direct interference by Rep had been controlled for, by co-transfecting the Rep expression vector (pTHRep) with the luciferase positive control (pGI4.13), and no significant change in luminescence was observed (FIG. 3A).

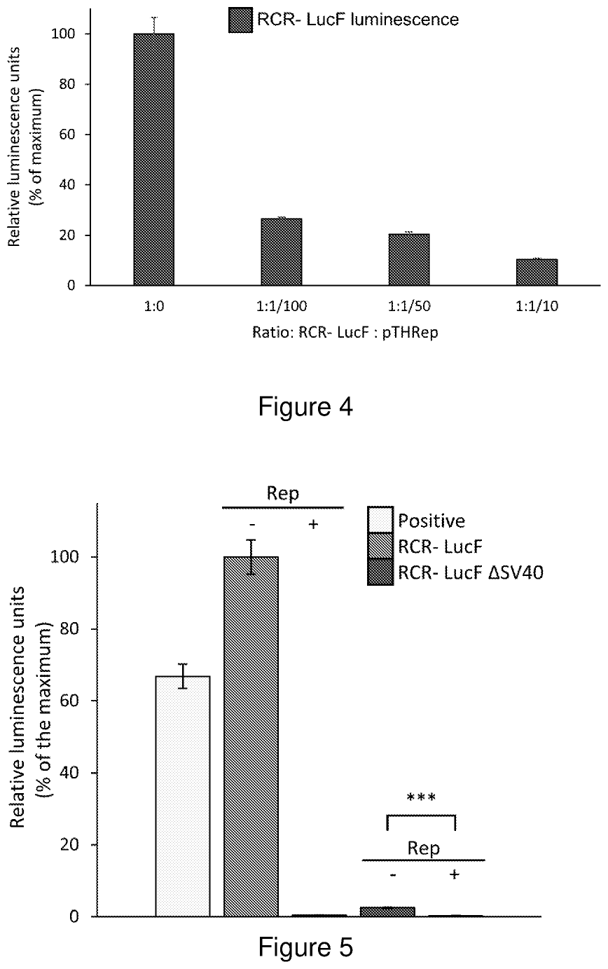

To further investigate the effect of rep on luminescence when co-transfected with RCR− LucF, a series of dilutions were set up to titrate out expressed Rep (FIG. 4). Luminescence was observed to be inversely proportional to the amount of rep present from pTHRep co-transfections, demonstrating that lowering Rep concentration increased luminescence (FIG. 4). This confirmed that the overexpression of Rep was responsible for reducing luminescence, and that this inhibition was linked to the presence of the BFDV ori. Rep is known to bind a repeated octa-nucleotide motif (5′-GGGCACCG-3′; SEQ ID NO:10) situated within the ori. Binding these sequence repeats is a critical step in RCR initiation and shared among all circoviruses. Rep binding here is crucial in destabilising the dsDNA and allowing the formation of a stem-loop structure that is nicked and unravelled during RCR. In this process, the viral genome can self-prime and recruits host cell DNA polymerase to carry out its genomic replication cycle via RCR. Rep competes directly with RNA transcriptase for DNA binding space, as the promotor for Rep is also located within the ori, immediately downstream of the Rep binding site. This creates a dynamic, self-regulatory effect on Rep expression, which decreases as its saturation level increases. This, however, was not expected to directly affect luciferase expression, as that was driven by the SV40 enhancer/promoter sufficiently downstream of the ori, nevertheless when the vector is undergoing RCR expression could still be disrupted.

Similar inhibitory effects have been observed in other related circoviruses such as PCV, where it has been suggested that the inhibitory effect is also linked to the ability of PCV Rep to interact/recruit the activity of transcription inhibitors and other host proteins. As a DNA binding protein, and one that is capable of recruiting transcription inhibitors, Rep may have the ability to disruptively affect host cell gene expression as well. This ability might constitute a form of viral defence, whereby disruptive inhibition of host genes, particularly antiviral response genes, may occur if Rep recognises any transcriptionally active host cell DNA sequences. Preliminary work done assessing antiviral response genes indicated that Caspase 10 might be one such gene as it was strongly downregulated compared to a pUK19 control in cells where Rep was overexpressed.

### Example 3

Evaluation of Strength of the Native Rep Promoter within the Ori

To evaluate the strength of the native rep promoter within the ori, the SV40 promoter was removed from RCR− LucF to create the vector RCR− LucF ΔSV40. Luminescence from both vectors was compared in the presence and absence of Rep (FIG. 5). This time the positive control was transfected at the same copy number as the RCR− LucF vector to investigate whether the ori was acting as an enhancer element to the SV40 promoter. The results indicated an increased luminescence of approximately 50% over the positive control under the tested conditions (FIG. 5). When luciferase expression was driven by the native rep promoter using RCR− LucF ΔSV40, luminescence was 3.8% of that achieved by the positive control (SV40). As previously demonstrated, luminescence was drastically reduced in the presence of Rep (p<0.001). Thus, Rep's native promoter expressed luciferase at approximately 3.8% of the level obtained using the SV40 promoter. Interestingly it appears the native promoter also had an enhancing effect when situated immediately upstream of the SV40 promoter/enhancer of around 50%. This confirms that pTHRep, a potent mammalian gene expression vector, for transient Rep expression, results in prohibitive Rep overexpression and is likely the direct cause of decreased luciferase expression.

To evaluate the effect of Rep provided in cis with its native promoter, the luc cassette was placed downstream of the viral ori rep sequence to create the RCR+ LucF vector. Two fully self-replicating vector pairs (RCR+ LucF/R) and an additional non-replicating control (RCR− LucR) were developed for this purpose. These new vectors expressed Rep under its native viral promoter. In addition to this, the luciferase expression cassette was tested downstream of Rep on the same sense strand in a forward orientation (F) or on Reps antisense strand in a reverse orientation (R), truncating the majority of the BFDV capsid gene (FIG. 1). The creation of RCR amplicons was verified as before using RCA, and gene of interest expression level evaluated using luciferase assay.

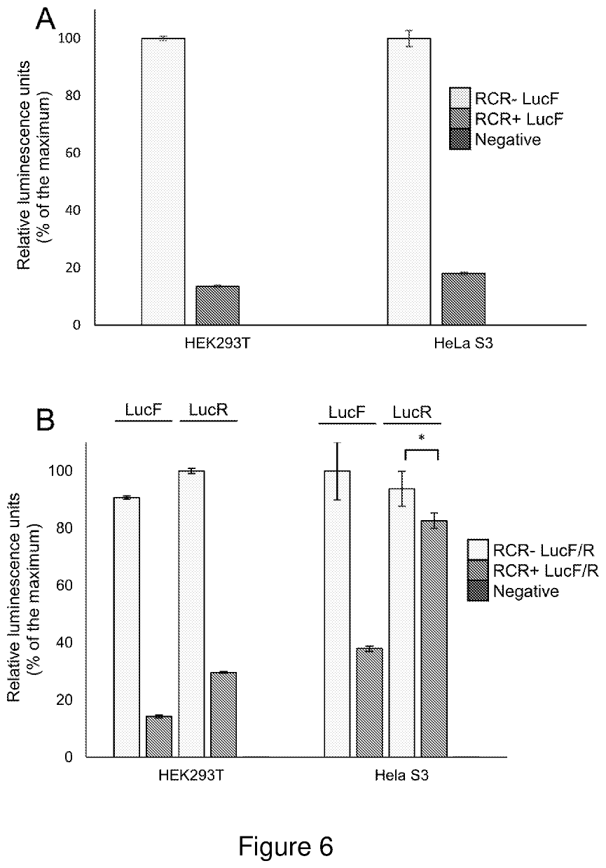

Luminescence was compared with RCR− LucF in HEK293T and HeLa S3 cells. Luminescence readings from RCR+ LucF improved in both HEK293T and HeLa S3 cells yet were still well below the levels obtained by the non-replicating RCR− LucF control (FIG. 6A). This indicated that luciferase expression was still being suppressed, albeit less than before (FIG. 5). Thus, in each cell line, luminescence decreased in the presence of Rep under control of its native promoter; however, the reduction was less noticeable than when Rep was supplied in trans.

To determine whether the interaction of Rep with the upstream ori was responsible for observed decreases in luminescence, the luc expression cassette was placed on the antisense strand to create the RCR− LucR and RCR+ LucR vectors. Luminescence was then compared between these two vectors in HEK293T and HeLa S3 mammalian cells. A comparison in HEK293T mammalian cells demonstrated a limited improvement when compared to the luc cassette placed on the sense strand. Surprisingly, when the luc expression cassette was placed antisense to Rep, luminescence was significantly (p<0.001) improved in HEK293T cells and almost fully restored to the level obtained from the non-replicating RCR− LucR control in HeLa S3 cells (FIG. 6 B). It is noted that in this configuration, the luc expression cassette is situated in the location of the truncated BFDV capsid gene. This improved expression could be attributed to evolutionary pressures requiring higher viral capsid gene expression levels.

The observed differences in luminescence between HEK293T and HeLa S3 cells (FIG. 6 B) further demonstrates the dynamic nature of the inhibition generated by Rep during RCR. It is noted here, however, that HEK293T cells contain the large T-antigen from the simian virus 40, which enhances plasmid DNA transfer to the nucleus from the cytoplasm. This greatly improves nuclear localisation and gene expression from transfected DNA but may affect the delicate balance between Rep and its self-regulated RCR cycle. Such an artificial enhancement works well in tissue culture to improve gene expression from DNA vectors but badly represents conditions experienced in-vivo for DNA vaccines. This also offers a plausible explanation of why the self-replicating RCR+ LucR vector's performance was more comparable to its non-replicating twin (RCR− LucR) in HeLa S3 cells. Furthermore, HeLa S3 cells offer none of the artificial enhancement provided by the SV40 large T antigen. For this reason, they were considered as a better cell culture testing model for extrapolating how vaccines, developed using this technology, would perform in animal models.

### Example 4

Comparison of Replication-Competent BFDV Expression Vector with Non-Replicating Vectors

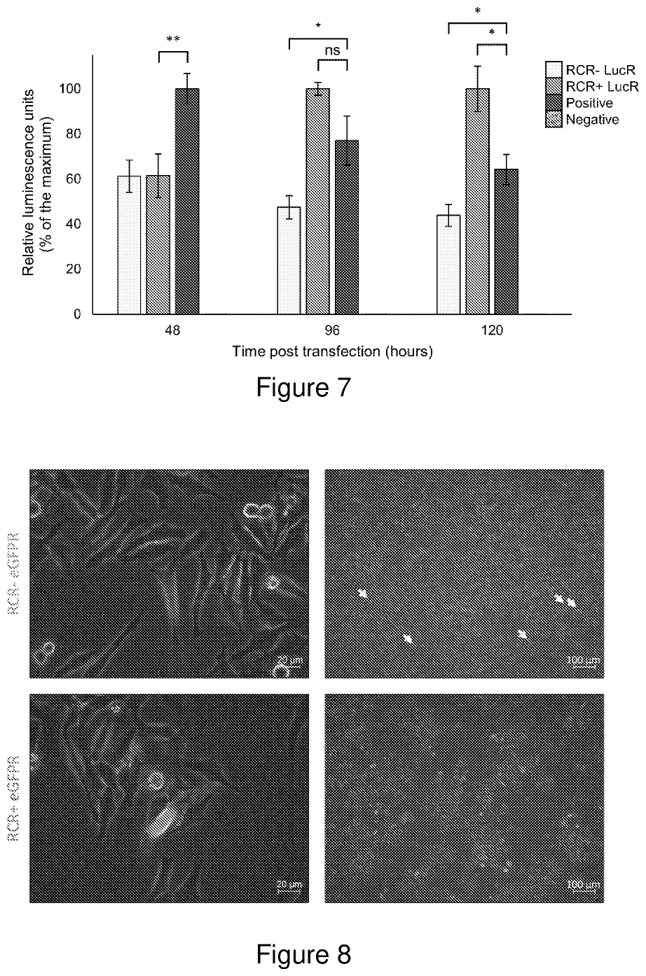

A comparison was made over time between the RCR− LucR, RCR+ LucR (containing the luc cassette on the antisense strand) and the positive control. Less luminescence was observed over time for non-replicating luciferase expression vectors RCR− LucR and positive control. No difference in luminescence was observed between RCR− LucR and RCR+ LucR at 48 hours post-transfection; however, at 96 hours post-transfection (including 1 passage) luciferase luminescence from the replicating RCR+LucR surpassed the levels from both the RCR− LucR and positive control vectors (P<0.05) with luminescence among HeLa S3 cells transfected replicative RCR+ LucR approximately 2-fold higher compared to non-replicative RCR− LucR transfected cells. At 120 hours post-transfection, these differences increased further (P<0.05) (FIG. 7). This demonstrated that replicative RCR+ gene expression vectors could maintain higher gene expression levels over time.

To further investigate the differences between the replicating and non-replicating vectors and to quantify gene expression at the cellular level, the luc reporter was replaced with egfp to create RCR− eGFPR and RCR+ eGFPR (FIGS. 1 and 2). Transfected Hela S3 cells were sorted using FACS and re-cultured for up to nine days post-transfection. Fluorescing cells were viewed using inverted microscopy. Replication competent RCR+ eGFP transfected cells maintained highly elevated levels of eGFP expression over the entire duration of this experiment, which included 2 passages post-transfection. Fluorescence observations during this time were consistently high for the entire 9 days and across the whole RCR+ eGFP transfected cell population. This fluorescence was comparable to the brightest levels seen from non-replicative RCR− eGFP transfected cells at their peak (48 h post-transfection). Another intriguing observation was that RCR+ eGFP transfected cells appeared to exhibit a rounder cell morphology, possibly indicative of increased cellular stress. FACS data from cell sorting also revealed significant (P<0.001) increases in internal cellular granularity and cell size among RCR+ eGFP transfected cells. Non-replicative RCR− eGFP transfected cells, however, exhibited a typical gene expression profile as measured by fluorescence, peaking in brightness from 48-72 hours post-transfection and fading after that (FIG. 8).

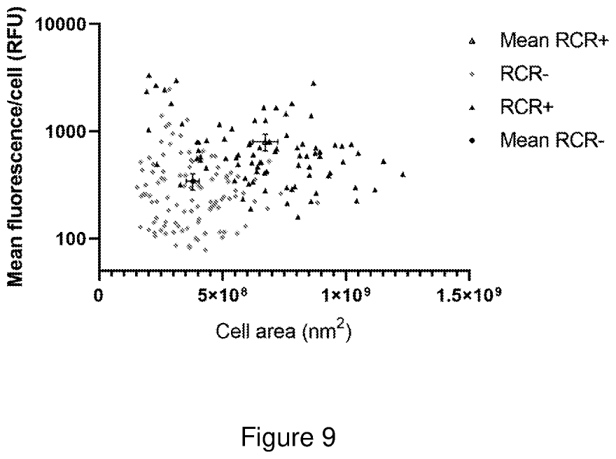

To quantify the amplification of fluorescence and cell size changes, replicating and non-replicating eGFP vectors were transfected into HeLa S3 cells using an optimised transfection protocol. This was designed to minimise DNA vector uptake to mirror DNA vaccine uptake observed in live animal models. Fluorescence and cell size were quantified using confocal microscopy, 24 hours post-transfection (FIG. 9). The results indicate that the amplification effect, achieved by RCR, occurs rapidly and is more prominent when starting with a lower DNA transfection level. When evaluating individual cells, DNA transfection occurs across a distribution of concentration levels, resulting in a distribution of observed cellular fluorescence levels. This is logical as some cells may uptake more DNA during transfection than others. Such a distribution is seen for both the RCR+ and RCR− eGFP samples, however for the RCR+ eGFP transfected cells, this entire distribution appears to have been effectively shifted upwards by a mean factor of 2.3-fold, indicating an increased in mean cellular fluorescence intensity per cell (FIG. 9). Similarly, a distribution in cell size is also present and expected among cells, which can be attributed to the normal cell growth and division cycles. Interestingly RCR+ eGFP transfected cells were seen to have also shifted their size distribution range, to be, on average, larger by a factor of 1.8-fold while also extending the overall cellular size range (FIG. 9). Because the fluorescence measurement made by the confocal microscope is recorded as mean cellular fluorescence per area, this size shift would also correlate with overall more eGFP per cell because cell size and volume are no longer directly comparable between the RCR+ and RCR− eGFP transfected cells.

These size changes suggest complex internal cellular biochemistry and alterations in internal homeostasis are occurring. Literature on RCR indicates that the process is prominent when a host cell enters S-Phase. Seemingly, when the host cellular machinery necessary for RCR, such as DNA polymerase, is most readily available. This idea is supported by the observed increase in cellular granularity, which is known to correlate with changes in DNA conformation and concentration, that was seen among RCR+ eGFP transfected cells during FACS. Whether RCR or Rep can induce entry into S-Phase and maintain or halt it in this state are intriguing questions worthy of further investigation. Regardless, a robust innate cellular and antiviral gene response are expected from RCR because of the stimulation that active, pathogenic genomic replication can have on known viral PRRs. Recent research has indicated that pathogen viability and threat level is at least in part determined by quantifiable detection of foreign genomic replication and gene expression. These signals can be used by the immune system to inform the magnitude and type of immune response required to address the threat resulting in the generation of a unique PAMP. The potential for RCR amplified gene expression vectors to directly trigger known innate PRRs and activate antiviral cellular responses would be a beneficial research and vaccine design tool.

### Example 5

### Expression in Mouse Animal Experimental Model

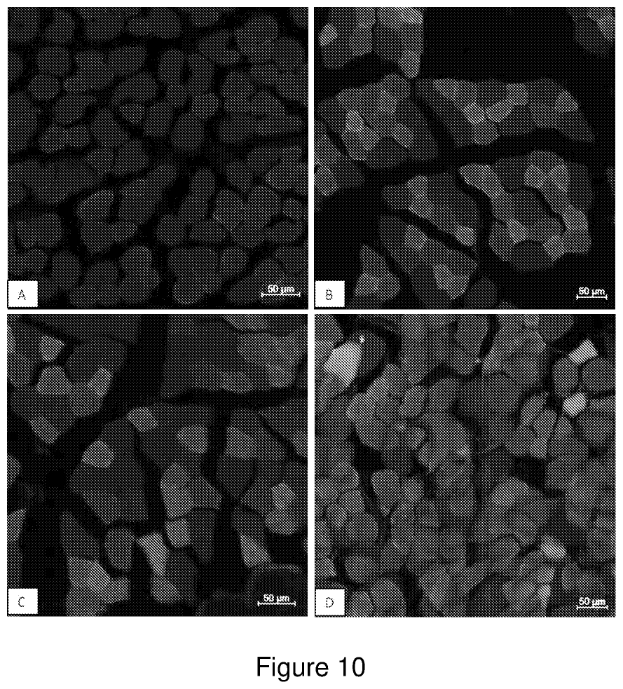

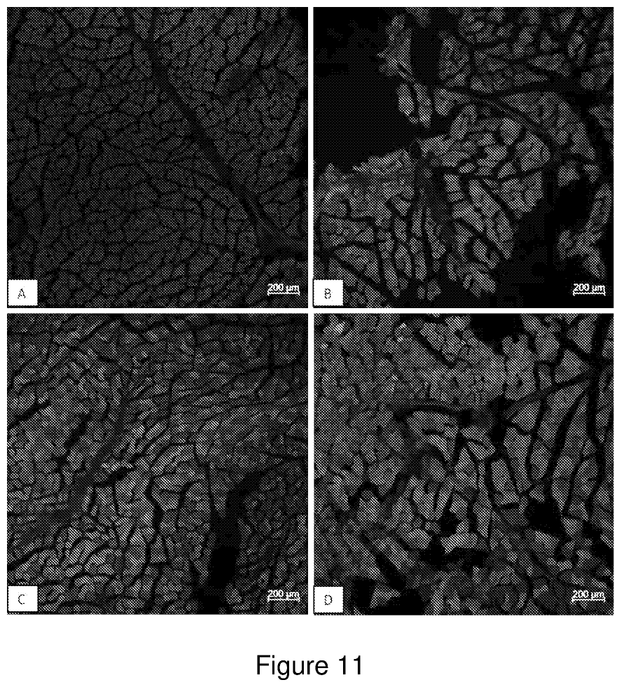

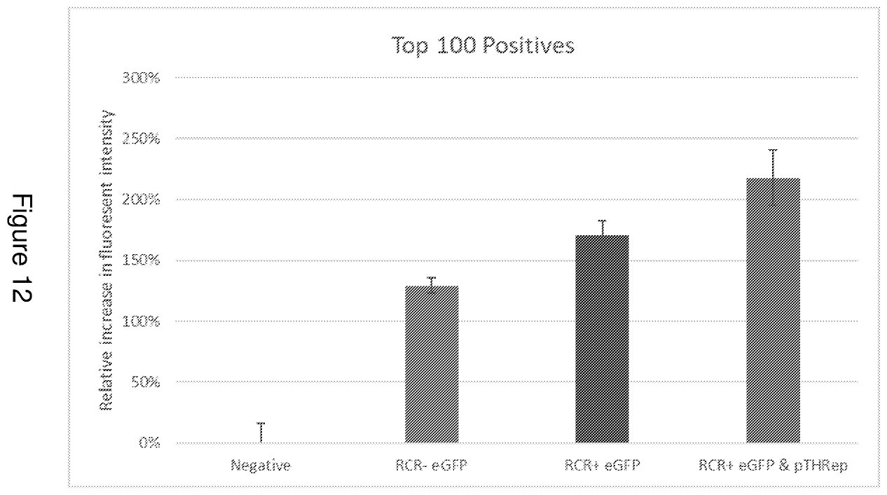

Confocal microscopy was used to quantify eGFP expression in mouse muscle cross sections following inoculation to demonstrate the expression of the self-replicating vectors within a mouse animal experimental model. Tissue sections of negative control mouse samples indicated a uniform level of eGFP fluorescence from all muscle fibre bundles as expected. This was recorded and used to define the cut-off threshold for positive fluorescence detection. The fluorescence of the RCR+ eGFP transfected muscle samples and that of the RCR+ eGFP and pTHRep boosted sample both exhibited brighter eGFP fluorescence than the RCR− eGFP samples 28 days post inoculation. The presence of a few exceptionally bright muscle fibre bundles was noted only in the RCR+ eGFP with pTHRep boosted samples (FIGS. 10 and 11).

To capture as much image data as possible from each anterior tibialis muscle cross section, tile scan images of up to 8×8 Z-stack captures were taken and merged. These images gave a good overview over the whole muscle cross section: these merged images are depicted in FIG. 11. Overall, it was evident that for the RCR− samples there was noticeably more fluorescence, but this was uniform in nature. In contrast to that the RCR+ vector inoculated samples clearly exhibited greater variance in fluorescence brightness between individual muscle fibre bundles (FIG. 11).

To quantify gene expression in the mouse muscle tissues, confocal microscopy was used to evaluate the relative mean eGFP fluorescence of individual muscle fibre bundles from the different inoculation groups of mice (FIG. 12). The mean fluorescence of the brightest 100 muscle fibre bundles was recorded from each mouse muscle tissue sample and normalized by subtracting the mean background/autofluorescence signal to determine a true signal. These data were then depicted as a relative percentage change and their significance was calculated by ANOVA using (p=0.5). A minimum signal cut-off value was also set as two standard deviations (2σ) of the negative control, being 20%. This analysis indicated that the RCR− eGFP sample had a mean eGFP fluorescence level in affected muscle fibre bundles that was 129+/−5% brighter than the background/autofluorescence level. Tissue inoculated with RCR+ eGFP vectors were ˜170% brighter than negative/background levels, and tissues inoculated with both RCR+ eGFP and pTHRep were ˜217% above background levels (FIG. 12). The standard error of the mean fluorescence from between the three tissue samples from each mouse was calculated for FIG. 12 to be +/−6% for the RCR− sample +/−16% for the RCR+ sample and ˜+/−23% for the RCR+ eGFP and pTHRep sample. An ANOVA was also performed for all 300 readings made (100 per sample) which indicated the differences in means were highly significant (p>0.99). Changing variance and fluorescence range between samples was also observed.

### REFERENCES

Bai, H., Lester, G. M. S., Petishnok, L. C. & Dean, D. A. Cytoplasmic transport and nuclear import of 516 plasmid DNA. Biosci Rep 37 (2017).
Regnard, G. L., de Moor, W. R. J., Hitzeroth, II, Williamson, A. L. & Rybicki, E. P. Xenogenic rolling-circle replication of a synthetic beak and feather disease virus genomic clone in 293TT mammalian cells and Nicotiana benthamiana. J Gen Virol 98, 2329-2338 (2017).

1. A self-replicating DNA expression vector comprising:
a) at least one BFDV LIR element comprising an origin of replication (Ori);
b) a gene encoding a BFDV replication-associated protein (Rep);
c) a gene encoding a heterologous polypeptide of interest; and
d) a mammalian promoter operably linked to (b) and (c),
wherein the Rep protein is capable of binding to the Ori to initiate replication of the expression vector.

2. The self-replicating DNA expression vector of claim 1, further comprising a vector backbone.

3. The self-replicating DNA expression vector of claim 1, further comprising a second BFDV LIR element.

4. The self-replicating DNA expression vector of claim 1, wherein (a) and (b) are in a forward orientation on the vector.

5. The self-replicating DNA expression vector of claim 1, wherein (c) and (d) are in reverse orientation on the vector.

6. The self-replicating DNA expression vector of claim 1, wherein the mammalian promoter is a simian vacuolating virus 40 promoter (SV40).

7. The self-replicating DNA expression vector of claim 1, wherein the vector has a truncated or absent BFDV capsid protein gene.

8. The self-replicating DNA expression vector of claim 1, further comprising one or more restriction enzyme recognition sites.

9. The self-replicating DNA expression vector of claim 1, further comprising a partial Kozak sequence, optionally having a nucleic acid sequence of SEQ ID NO:5.

10. The self-replicating DNA expression vector of claim 1, wherein the vector is capable of replicating to high copy number and remaining as an episome.

11. A pharmaceutical composition comprising the self-replicating DNA expression vector of claim 1 and a pharmaceutically acceptable diluent or excipient.

12. The pharmaceutical composition of claim 11, wherein the pharmaceutical composition is capable of eliciting a protective immune response in a subject against a disease.

13. The pharmaceutical composition of claim 11, which is formulated for intramuscular, intradermal, intravenous or subcutaneous administration.

14. A cell comprising the self-replicating DNA expression vector of claim 1.

15.-18. (canceled)

19. A method of inducing an immune response in a subject comprising administering the self-replicating DNA expression vector of claim 1 to the subject.

20. The method of claim 19, wherein the immune response is a neutralizing antibody response or a cytotoxic T lymphocyte response.

21. A method of expressing a polypeptide in a subject comprising administering the self-replicating DNA expression vector of claim 1 to the subject.

22. The method of claim 19, wherein the subject is a human.

23. An in vitro or ex vivo method of expressing a polypeptide in a cell comprising transfecting the cell with the self-replicating DNA expression vector of claim 1.

24. The method of claim 23, further comprising recovering the polypeptide from the cell.

25. The method of claim 21, wherein the subject is a human.


SEQUENCE LISTING (9 sequences)

SEQ ID NO: 1 - DNA, 160 bp, Beak and Feather Disease Virus
  source 1..160: mol_type = other DNA
agaggtgccccacaggcggcggttagtattacccgccgcctggggcaccggggcaccgcagccattggctgccgtgccgaggtgccccgccttagggaggagtaaatggcgccgttaaacggtgccgtaatttccggaggatcacagtcgcccgggaaca

SEQ ID NO: 2 - DNA, 867 bp, synthetic construct
  misc_feature 1..867: note = BFDV Rep gene
  source 1..867: mol_type = other DNA
atgccgtccaaggagggctctggctgtcgccgttggtgtttcacccttaacaaccctacagacggcgagatcgaattcgtccgtactctcgggcctgacgaattctactatgccatcgttggacgggaaaagggcgagcaaggtaccccccatttgcaaggctactttcatttcaaaaataagaagcgactgagcgcgcttaataaaatgctgccgcgagctcattttgagcgcgctaaagggagtgatgcagataatgagaagtattgcagtaaagagggggacgttatacttaccctgggcattgtggcgagagatggtcaccgcgctttcgacggagctgttgctgccgtgatgtccggacgcaaaatgaaggaagtcgcgcgagagttcccagatatctacgtcaggcatgggcggggcttgcataacctctctctattggtcggttcccgcccacgtgatttcaagacagaagttgacgtcatctacgggcctcctgggtgtggcaagagtaaatgggccaatgagcagcctgggactaaatattataaaatgcgcggtgaatggtgggatggatatgatggggaagatgttgtcatattggacgacttttatgggtggctaccttattgtgagatgctccgcctttgcgaccgttatccacataaagtgccagttaagggcgcttttgtggagtttaccagcaagaggatcattatcacgagcaataagtcccccgagacctggtacaaggaggactgtgacccgaagccactgttccggagattcactcgtgtttggtggtacactgacaagttggaacaagtccggcctgatttccttgcccaccccatcaatttttga

SEQ ID NO: 3 - protein, 288 aa, Beak and Feather Disease Virus
MPSKEGSGCRRWCFTLNNPTDGEIEFVRTLGPDEFYYAIVGREKGEQGTPHLQGYFHFKNKKRLSALNKMLPRAHFERAKGSDADNEKYCSKEGDVILTLGIVARDGHRAFDGAVAAVMSGRKMKEVAREFPDIYVRHGRGLHNLSLLVGSRPRDFKTEVDVIYGPPGCGKSKWANEQPGTKYYKMRGEWWDGYDGEDVVILDDFYGWLPYCEMLRLCDRYPHKVPVKGAFVEFTSKRIIITSNKSPETWYKEDCDPKPLFRRFTRVWWYTDKLEQVRPDFLAHPINF

SEQ ID NO: 4 - DNA, 358 bp, Simian Vacuolating Virus 40
  source 1..358: mol_type = other DNA
ctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 5 - DNA, 13 bp, synthetic construct
  misc_feature 1..13: note = Partial Kozak sequence
  source 1..13: mol_type = other DNA
gccaccatggcgg

SEQ ID NO: 6 - DNA, 31 bp, synthetic construct
  misc_feature 1..31: note = BFDV rep sense primer
  source 1..31: mol_type = other DNA
ggtggtgcggccgcagaggtgccccacaggc

SEQ ID NO: 7 - DNA, 30 bp, synthetic construct
  misc_feature 1..30: note = BFDV rep antisense primer
  source 1..30: mol_type = other DNA
ggtggtctcgagtcaaaaattgatggggtg

SEQ ID NO: 8 - DNA, 29 bp, synthetic construct
  misc_feature 1..29: note = egfp sense primer
  source 1..29: mol_type = other DNA
gtggtaagcttatggtgagcaagggcgag

SEQ ID NO: 9 - DNA, 35 bp, synthetic construct
  misc_feature 1..35: note = egfp antisense primer
  source 1..35: mol_type = other DNA
ggtggtggatccttacttgtacagctcgtccatgc